(19) United States
(12) Patent Application Publication
(10) Pub. No.: US 2017/0143545 A1
(43) Pub. Date: May 25, 2017

(54) EYEWEAR ASSEMBLY

(71) Applicants: Floris Zwolsman, Amsterdam (NL); Maarten Roodenburg, Amsterdam (NL)

(72) Inventors: Floris Zwolsman, Amsterdam (NL); Maarten Roodenburg, Amsterdam (NL)

(21) Appl. No.: 14/945,728

(22) Filed: Nov. 19, 2015

(51) Int. Cl. A61F 9/02; A42B 3/18

(52) U.S. Cl. CPC A61F 9/025; A61F 9/027; A42B 3/185

(57) ABSTRACT

A goggle comprising a frame with one or more openings; a first lens is having a front surface and a rear surface; a lens bracket having a front surface adapted to be coupled to the rear surface of the first lens; a second lens is having a front surface and a rear surface. The front surface of the lens bracket is coupled to the rear surface of the second lens such that the second lens is placed over the first lens, thereby forming a single consolidated lens. A plurality of connectors on the lens bracket are adapted to releasably couple with a respective plurality of coupling points formed on a front surface of the goggle. The lens is adapted to be releasably attached to the goggle by the lens bracket.

### TECHNICAL FIELD

The present subject-matter generally relates to a goggle, and more specifically to a goggle with releasably attached lenses with aid of magnetic coupling.

### BACKGROUND

With the passage of time, goggle have changed in terms of look and functional attributes to enable a wide range of users including skiers, motor-bike riders etc. There are further developed goggles which enable the user to replace the mounted lens. Such developed goggles may have more than one viewing lens coupled together.

Wearing such goggle and participating in various sports activities leads to fogging and smudges on the lens of the goggle. Conventional replaceable lens technology is difficult to manipulate, especially when the user in on the field and he/she has to replace lens with a gloved hand or dirty hand while in the field/ski slope. Such activity requires the user to remove the glove first and then change the lens or leave the field/ski slope to change the lens. This also results in a decreased peripheral viewing ability by not being able to quickly clean the (inside) of the lens of smudges or fog. Additionally, some lenses offer magnetic lens technology, but since the magnetic field and the number of magnetic points of such replaceable lens is low, this results in instances wherein such lens falls down in a process of replacing the lens by a user, or in case of a fall down by the user himself. Also, the magnetic force between the lenses may fade off due to regular wear and tear.

Therefore, an enhanced user friendly goggle is needed to address the aforementioned problems.

### SUMMARY

In one embodiment, a goggle is disclosed. The goggle comprising a frame with one or more openings; a first lens is having a front surface and a rear surface; a lens bracket having a front surface adapted to be coupled to the rear surface of the first lens; a second lens is having a front surface and a rear surface. The front surface of the lens bracket is coupled to the rear surface of the second lens such that the second lens is placed over the first lens.

In an another embodiment, a plurality of connectors on the lens bracket are adapted to releasably couple with a respective plurality of coupling points formed on a front surface of the goggle. The second lens is adapted to be releasably attached to the goggle by the lens bracket.

In an embodiment of the present invention, it is disclosed that front surface of the frame has a second portion that is recessed from a first portion. Plurality of coupling points is moulded on the second portion. The front surface of the first lens is proximate to the rear surface of the second lens and the rear surface of the second lens is proximate to the front surface of the first lens.

In an embodiment, a lens assembly is disclosed. The lens assembly with a first lens having a front surface and a rear surface; at least one lens bracket having a front surface adapted to be coupled to the rear surface of the first lens; a second lens having a front surface and a rear surface. The front surface of the lens bracket is coupled to the rear surface of the second lens such that the second lens is placed over the first lens and the first lens and the second lens is proximate to each other.

In yet an another embodiment, the plurality of connectors are placed at the outer edge of the lens bracket, wherein the outer edge is the portion of the lens bracket which touches the outer edge of the first lens and the second lens.

The device disclosed herein may be implemented in any means for achieving various aspects and may be implemented to perform in various ways. Other features will be apparent from the accompanying drawings and from the detailed description that follow.

### BRIEF DESCRIPTION OF THE FIGURES

Various embodiments are described herein with reference to the drawings, wherein:

FIG. 1 illustrates a top elevation view of the lens assembly;

FIG. 2 illustrates perspective view of the goggle showing outer edges of the lens bracket, in accordance with an embodiment of the present subject matter;

FIGS. 3A and 3B illustrate coupling of the magnetic connectors and points of the lens bracket and lens, in accordance with an embodiment of the present subject matter;

FIGS. 4A and 4B illustrate placement of lenses in the lens bracket and their magnetic coupling with respect to the lens bracket in accordance with an embodiment of the present subject matter; and

FIGS. 5A and 5B illustrate goggle with the lens assembly and strap in accordance with the present subject-matter.

The drawings described herein are for illustration purposes only and are not intended to limit the scope of the present disclosure in any way.

### DETAILED DESCRIPTION

In the following detailed description of the embodiments of the present subject matter, references are made to the accompanying drawings that form a part hereof, and in which are shown by way of illustration specific embodiments in which the present subject matter may be practiced. These embodiments are described in sufficient detail to enable those skilled in the art to practice the present subject matter, and it is to be understood that other embodiments may be utilized and that changes may be made without departing from the scope of the present subject matter. The following detailed description is, therefore, not to be taken in a limiting sense, and the scope of the present subject matter is defined by the appended claims.

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

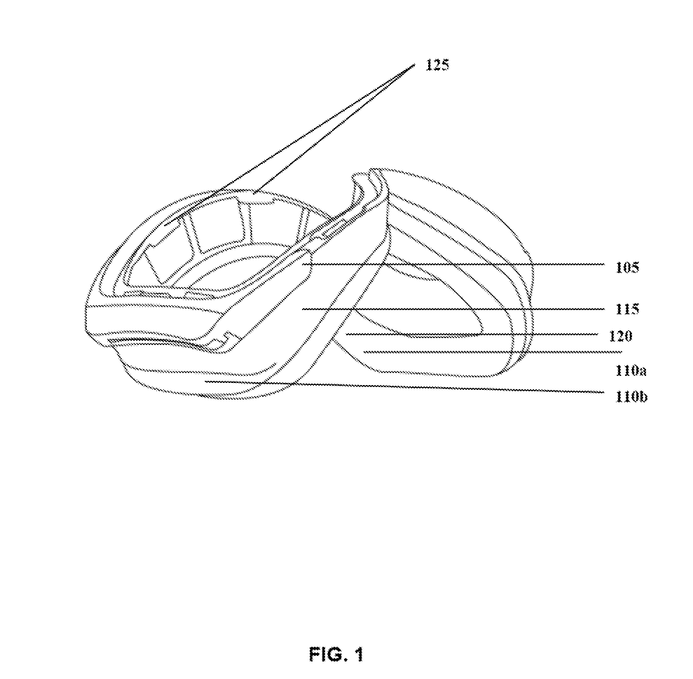

As illustrated in FIG. 1, the lens assembly 100 comprises a lens bracket 105 which is substantially curvilinear. This substantially curvilinear shape of the lens bracket 105 allows the lens assembly 100 to wrap around at least a portion of the user's face to provide the user with an enhanced viewing range. Although, it should be appreciated that angled and flat shaped lenses may also be used in the lens bracket 105.

The lens bracket 105 has two surfaces, namely outer surface 110a and inner surface 110b. Both of the surfaces of the lens bracket may be made of any suitable synthetic plastic, which has low elasticity. The outer surface 110a of the lens bracket 105 has two extremities which are diverged in to two segments to accommodate more flexibility to a user. These two segments are jointly held by the central portion of the lens bracket. A slab 115 of predefined structure of porous substance is sandwiched between these two joint segments. Further, the inner surface 110b of the entire lens bracket 105 with these two segments is mounted over another slab 120 of same or similar porous materials of predefined structure using some adhesive substance. Inclusion of such porous materials in the lens bracket enhances physical characteristics such as air ventilation, shock endurance, flexibility etc. There is plurality of magnetic connectors 125 at the outer edge of the lens bracket 105 which accommodates a lens with effect of magnetic coupling. In this regard, the plurality of magnetic connectors 125 of the lens bracket 105 should be in in the same coplanar axis of the similar structure of the lens to be accommodated.

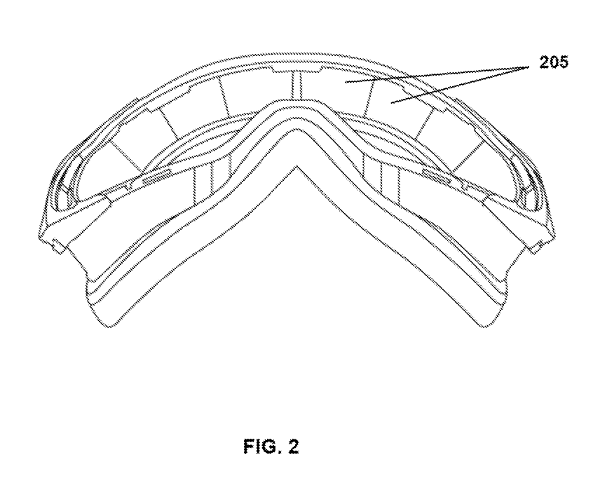

Referring now to FIG. 2, which illustrates sectional view of the lens assembly 100 showing insertion of lens(s), in accordance with an embodiment of the present subject matter. The lens assembly has a series of spacing apparatus or spacer 205 in between the longitudinal edges of the lens bracket 105. The spacing apparatus or spacer 205 of the lens bracket 105 couples to inner surface of an outer lens, for example, using an adhesive or other coupling means of the type. In an illustrative embodiment, the spacing apparatus 205 may be a foam tape or other structurally pliable material. The thickness of the spacing apparatus 205 may be about the same as the thickness of the lens bracket, allowing the inner lens to couple to the inner lens coupling surface of the spacing apparatus and to smoothly extend over portions of the lens bracket without creating substantial ridges or protrusions.

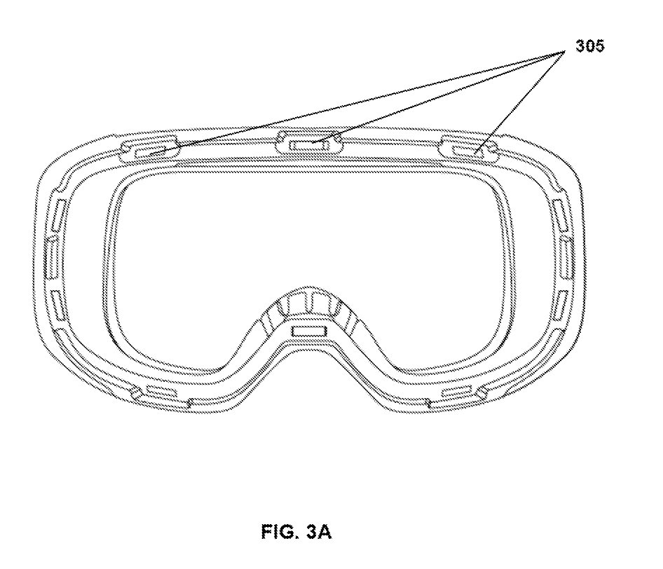

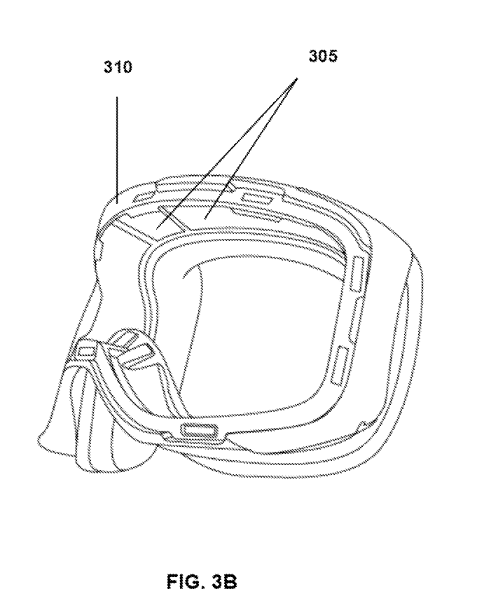

FIGS. 3A and 3B illustrate about other electronic components which may be associated with the lens assembly 100 and magnetic coupling between the lens bracket 105 and lens assembly 100. As per one embodiment of the present invention, there is plurality of magnetic connectors 305 at the outer edge of the lens bracket 105. These magnetic connectors 305 are placed apart in equal distance across the outer edge of the lens bracket 105. These magnetic connectors 305 are placed on the lens bracket 105 by means of moulded welding or the like. These magnetic connectors 305 serve the purpose of lens coupling in the lens assembly 100 as male interface.

In an another embodiment, one or more sensors may be placed on the lens assembly 100. The placement of such sensors may be outer edge of the lens bracket 105, or vicinity of the lens etc. The primary function of the sensors may be indicating the user that one or more lens of the goggle is not placed properly, indicating that the user need to clean the lens bracket 105 etc. Further, other components to support in performing the functions of sensors may also be present at the lens assembly 100.

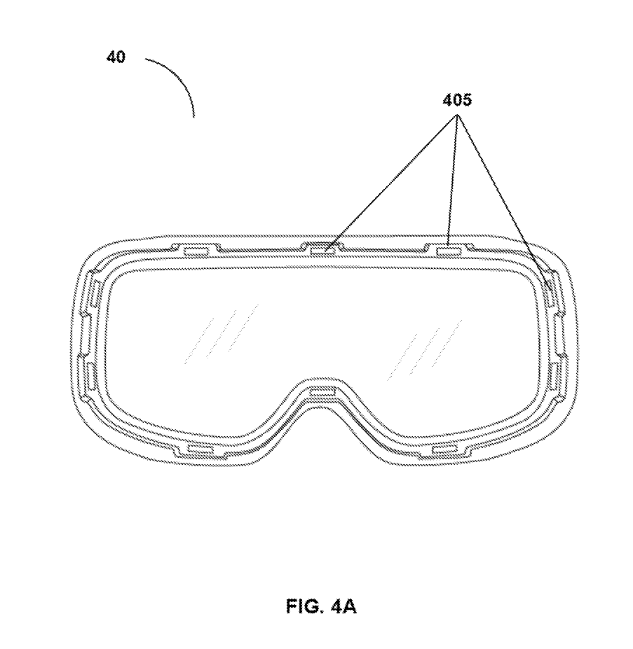

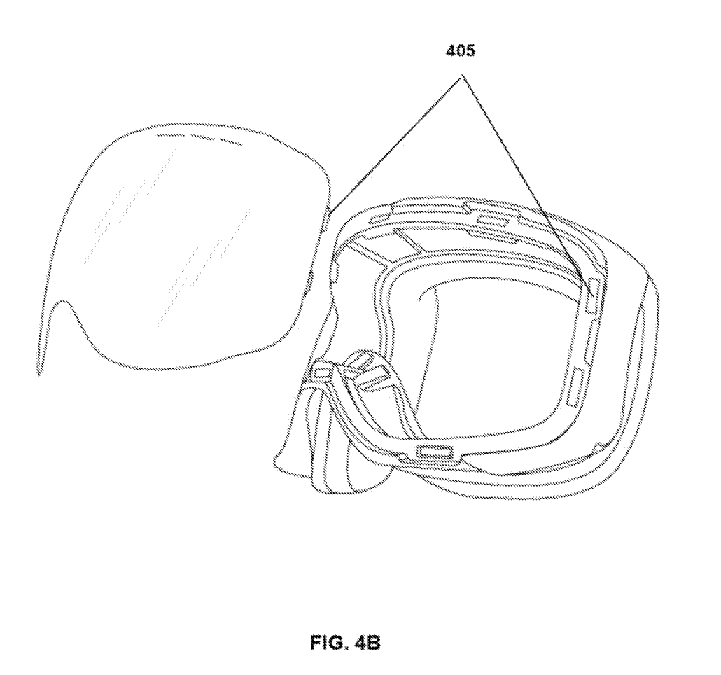

FIG. 4A illustrates about the magnetic coupling of a lens 400 with respect to an embodiment of the present invention. The lens 400 which is to be inserted will have plurality of coupling points 405. These coupling points 405 may be placed apart at the outer edge of the lens 400. These coupling points 405 are placed at the outer edge of the lens 405 by means of moulded welding or the like. These coupling points 405 are female interfaces. In operation, when a lens is put into the lens bracket 105, the lens 400 is placed well on the top of the plurality of connectors 125 of the lens bracket 105. It is to be mentioned that the plurality of connectors 125 of lens bracket 105 should be in same coplanar axis with that of the plurality of coupling points 405 in order to get the lens locked in the lens bracket 105 with the aid of magnetic force. In other words, it can be said that the position of connectors 125 and coupling points 405 should be in same place so that the lens can be placed in locking position with the help of coupling magnetic force.

FIG. 4B demonstrates that in order to pull out the lens from the lens bracket or change the lens from the lens bracket, a user has to hold the lens bracket with one hand and by another hand (with or without gloves) the entire lens can be held and taken out from the lens bracket. In similar fashion, a lens also can be inserted or put into the lens bracket.

For example, when the user has to put a lens to enhance further vision, then the user first has to remove the lens which is in order in the lens bracket. Upon removal of the lens, the user can put the lens into the lens bracket with ease. In such a process, the outer surface of the lens bracket includes one or more magnetic connectors and which are moulded on the outer edge of the lens bracket. Similarly, the one or more coupling points on the outer edge of the lens are also moulded and have to be on the same position with magnetic connectors when placed on the lens bracket.

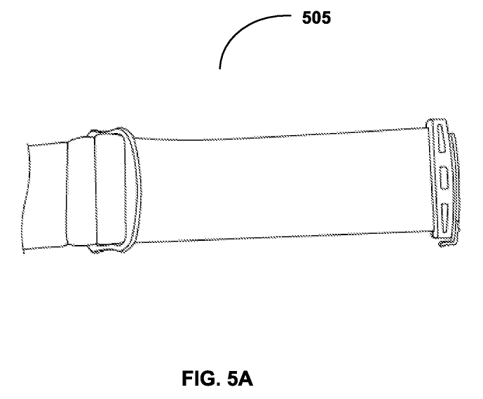

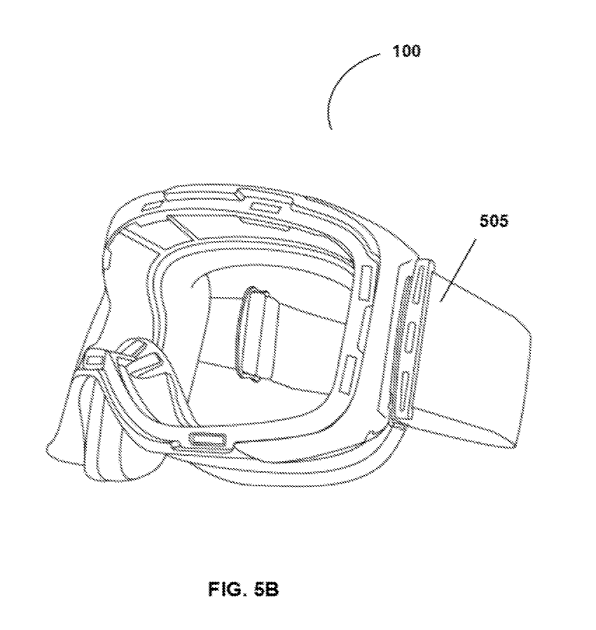

FIGS. 5A and 5B illustrate about placing the goggle on user's head securely. In an embodiment of the present invention, a goggle securing strap 505 may be attached to the lens assembly 100 or the lens bracket 105 to enable user to wear it around the head comfortably. Such strap may include other features, such as tension adjustment buckles, earstems that extend from the goggle to engage the sides and/or ears of a wearer, snaps, clips, hook-and-loop fasteners or other devices to secure the goggle to a helmet or other headgear, and so on. In addition to the above, in some embodiments the goggle may include adjustable arms located at the sides of the frame that the head support may be attached to. The adjustable arms may allow a user to adjust the fit of the goggle. The goggle as disclosed herein with replaceable lens may be used with any type of head support that allows the goggle to be retained on the head of an individual. Further, the strap 505 so attached may be developed to ease a user to attach/detach with the lens assembly 100 or the lens bracket 105. Also, the strap 505 may have other suitable features such as easy slide and lock etc. to serve the purpose quickly.

Although illustrative embodiments of goggle are described herein, it should be appreciated that in the development of one or more actual embodiments, numerous implementation-specific decisions may be made to achieve specific goals, for example, compliance with system-related and business-related constraints, which may vary from one implementation to another. Moreover, it should be appreciated that a development effort can be complex and time-consuming, but should nevertheless be a routine undertaking for one skilled in the art having the benefit of this disclosure.

Although the goggle have been described and illustrated in connection with certain embodiments, many variations and modifications should be evident to one skilled in the art and may be made without departing from the spirit and scope of the disclosure. The disclosure is thus not to be limited to the precise details of methodology or construction set forth herein as such variations and modification are intended to be included within the scope of the disclosure. Moreover, unless specifically stated any use of the terms first, second, etc. do not denote any order or importance, but rather the terms first, second, etc. are merely used to distinguish one element from another. Accordingly, the disclosure is limited only by the following claims.

In the foregoing description, certain terms have been used for brevity, clearness, and understanding. No unnecessary limitations are to be implied there from beyond the requirement of the prior art because such terms are used for descriptive purposes and are intended to be broadly construed. Therefore, the disclosure is not limited to the specific details, the representative embodiments, and illustrative examples shown and described. Thus, this application is intended to embrace alterations, modifications, and variations that fall within the scope of the appended claims.

Moreover, although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A goggle, comprising:
a frame with one or more openings;
a first lens having a front surface and a rear surface;
a lens bracket having a front surface adapted to be coupled to the rear surface of the first lens;
a second lens having a front surface and a rear surface, wherein the front surface of the lens bracket is coupled to the rear surface of the second lens such that the second lens is placed over the first lens, thereby forming a consolidated lens; and
a plurality of connectors on the lens bracket are adapted to releasably couple with a respective plurality of coupling points formed on the front surface of the lens bracket, wherein the lens is adapted to be releasably attached to the goggle by the lens bracket.

2. The goggle of claim 1, wherein front surface of the frame has a second portion that is recessed from a first portion, wherein the plurality of coupling points are formed on the second portion and wherein the front surface of the first lens is proximate to the rear surface of the second lens.

3. The goggle of claim 1, wherein front surface of the frame has a second portion that is recessed from a first portion, wherein the plurality of coupling points are formed on the second portion and wherein the rear surface of the second lens is proximate to the front surface of the first lens.

4. The goggle of claim 1, wherein the first lens rear surface and the second lens front surface are mounted on the lens bracket.

5. The goggle of claim 1, wherein the plurality of connectors combines the first lens, the second lens and the lens bracket together.

6. The goggle of claim 1, wherein each of the plurality of coupling points is an aperture like structure and is configured to releasably receive each of the plurality of connectors.

7. The goggle of claim 1, wherein the first and second lenses are injection molded lenses.

8. A lens assembly comprising:
a first lens having a front surface and a rear surface;
at least one lens bracket having a front surface adapted to be coupled to the rear surface of the first lens;
a second lens having a front surface and a rear surface, wherein the front surface of the lens bracket is coupled to the rear surface of the second lens such that the second lens is placed over the first lens and the first lens and the second lens is proximate to each other, thereby forming a consolidated lens; and
a plurality of connectors on the lens bracket are adapted to releasably couple with a respective plurality of coupling points formed on the front surface of the lens bracket , wherein the lens is adapted to be releasably attached to the goggle by the lens bracket.

9. The lens assembly of claim 8, wherein the plurality of connectors are placed at the outer edge of the lens bracket, wherein the outer edge is the portion of the lens bracket which touches the outer edge of the first lens and the second lens.

10. The lens assembly of claim 8, wherein the plurality of coupling points are present at the outer edge of the first lens and second lens.

11. The lens assembly of claim 8, wherein the plurality of connectors further comprises at least one magnet adapted to couple to a corresponding coupling point on the goggle by magnetic force.

12. The lens assembly of claim 8, wherein the plurality of connectors are with male interfaces and the coupling points are with female interfaces.
